(19) United States
(12) Patent Application Publication
(10) Pub. No.: US 2022/0172813 A1
(43) Pub. Date: Jun. 2, 2022

(54) AUTOMATIC TIME-BASED MEDICATION DISPENSER

(71) Applicant: PIYUSH SAKHARELIYA, EASTVALE, CA (US)

(72) Inventor: PIYUSH SAKHARELIYA, EASTVALE, CA (US)

(21) Appl. No.: 17/216,983

(22) Filed: Mar. 30, 2021

Related U.S. Application Data
(62) Division of application No. 17/106,945, filed on Nov. 30, 2020, now abandoned.

(51) Int. Cl. G16H 20/13; G06N 5/04; B65D 83/04; G06N 20/00

(52) U.S. Cl. CPC G16H 20/13; G06N 20/00; B65D 83/0454; G06N 5/04

(57) ABSTRACT

The present disclosure discloses a device for dispensing at least one substance. The device comprises a rotatable circular tray, a stationary circular plate, and a channel. The rotatable circular tray comprises a plurality of compartments. The plurality of compartments holds the at least one substance. The stationary circular plate is mechanically coupled below the rotatable circular tray. The stationary circular plate comprises an aperture. The channel is positioned below the aperture. The channel guides the at least one substance dispensed from the plurality of compartments to a collection tray. An identification sensor is affixed to the collection tray to acquire identification data associated with a patient and dispense the at least one substance when the identification data associated with the patient is recognized and matched. The device diverts the at least one substance missed by the patient from the collection tray to a disposal tray.

### CROSS-REFERENCE TO RELATED APPLICATIONS

This application is a divisional application of U.S. application Ser. No. 17/106,945 filed on Nov. 30, 2020, the contents of which are hereby incorporated by reference in their entirety.

### FIELD OF THE INVENTION

The present disclosure relates broadly to medication dispensing and more particularly to automatic time-based medication dispensing, preventing medication error and ensuring medication compliance.

### BACKGROUND

“An automatic pill dispenser for dispensing medical pills having different prescribed administration schedules includes a plurality of pill storage compartments each capable of holding more than one pill, an automatic release mechanism for dispensing pills at predetermined time intervals corresponding with their respective administration schedules, and a pill receptacle coupled to a pill detector such that a pill dispensed from the pill dispenser and received by the receptacle causes the pill dispenser to generate a signal to alert the patient to take the dispensed medicine. Twelve storage compartments, arranged in a ring about a vertically rotating wheel, are preloaded by a patient with all pills prescribed to be taken during a 24-hour period. The patient loads pills into individual storage compartments according to a loading code corresponding to the respective administration schedules of the pills. The pill dispenser then automatically sequentially rotates the storage compartments over a trapdoor which operates to empty each compartment positioned thereover. A photoelectric detector, having a light beam traveling vertically through a cup into which pills are dispensed, detects pills therein and responds by alerting the patient with an audible or visual signal. The light source also serves as a night light. A preloadable pill storage canister is also disclosed which may be preloaded by a pharmacist and simply inserted into the pill dispenser by the patient for automatic pill dispensing. A method of dispensing medical pills having different prescribed administration schedules is also described.” [Source: Automatic pill dispenser and method of administering medical pills, Kermit E. Lewis; Arthur S. Roberts, Jr.; published at USPTO as US4573606A on 4 Mar. 1986].

“A device and method for dispensing pills or vitamins is disclosed which includes a rotatable chamber within a housing. The chamber contains multiple slots for storing the pills or vitamins, and the housing has at least one dispensing hole so that pills will fall from the containment slot when it is aligned with the hole. The chamber may be rotated by motorized or manual means, with the preferred motorized means being an electric motor connected to a worm drive that engages gear teeth along the edge of the chamber, and the preferred mechanical means being a handle with a hinged tab.” [Source: Automatic pill dispenser, Kenneth Stillwell; Kenneth Stillwell, Jr; published at USPTO as U.S. Pat. No. 6,427,865B1, on 6 Aug. 2002].

Considering the knowledge of the persons skilled in the art, there is a long-felt need for a simple and automatic time-based medication dispenser that automatically prevents medication error, and assists in tracking and ensuring medication compliance.

### SUMMARY

The present disclosure discloses one or more aspects of automatic time-based medication dispensing and ensuring medication compliance.

In an aspect, a device is described herein. The device comprises a rotatable circular tray, a stationary circular plate, and a channel. The rotatable circular tray comprises a plurality of compartments. The stationary circular plate mechanically coupled below the rotatable circular tray. The stationary circular plate comprises an aperture. The channel is positioned below the aperture of the stationary circular plate. The channel guides at least one substance dispensed from the plurality of compartments to a collection tray.

In an embodiment, the aperture comprises surface area equal to surface area of at least one compartment of the plurality of compartments.

In another embodiment, the aperture comprises shape equal to shape of at least one compartment of the plurality of compartments.

In yet another embodiment, the device further comprises a sealing component between the rotatable circular tray and the stationary circular plate.

In yet another embodiment, the plurality of compartments comprises at least one of equal surface area and unequal surface area.

In yet another embodiment, the device further comprises a first sensor affixed to the collection tray recognizes identification data associated with a first user and operate the collection tray in response to recognizing the identification data.

In yet another embodiment, the first sensor comprises at least one of a radio-frequency identification (RFID) sensor, a biometric sensor, and a fingerprint sensor.

In yet another embodiment, the device further comprises a second sensor that detects whether the collection tray is operated within a predefined time from a scheduled time.

In yet another embodiment, the device further comprises a third sensor that acquires at least one physiological signal of a first user.

In yet another embodiment, the device further comprises a disposal tray positioned below the collection tray.

In yet another embodiment, the device further comprises one of (a) a permanent magnet direct current (PMDC) motor, (b) a stepper motor, and (c) a servo motor mechanically coupled to the rotatable circular tray.

In yet another embodiment, the device further comprises a microcontroller and a motor driver unit that actuates one of (a) the PMDC motor, (b) the stepper motor, and (c) the servo motor.

In yet another embodiment, one of (a) the PMDC motor, (b) the stepper motor, and (c) the servo motor rotates the rotatable circular tray sequentially and aligns at least one compartment of the plurality of compartments with respect to the aperture of the stationary circular plate.

In yet another embodiment, the device further comprises a fourth sensor affixed to the disposal tray that recognizes identification data associated with at least one of a second user, and a third user and operates the disposal tray in response to recognizing the identification data.

In yet another embodiment, the device further comprises a real-time clock electronically coupled with the microcontroller that actuates one of (a) the stepper motor, and (b) the servo motor at a scheduled time.

In yet another embodiment, the device is communicatively coupled to a server and a drug database.

In yet another embodiment, the device further comprises a camera and an artificial intelligence-based processing unit that determines whether a first user is accessing the device and collecting the at least one substance dispensed.

In yet another embodiment, the device communicates a signal to a server in response to the determination of whether the first user is accessing the device.

In another aspect, a system is described. The system comprises a server. The server comprises a memory, and a processor communicatively coupled to the memory, The processor is configured to provide a user interface to at least one of a first user, a second user, and a third user; receive at least one first input from at least one of the first user, the second user, and the third user via the user interface; receive at least one second input from a device, wherein the at least one second input comprises collection tray operating information, collection tray timing information, disposal tray operating information, disposal tray timing information, and physiological signal; analyze the at least one first input and the at least one second input received; and estimate and record at least one of medication compliance information, medication non-compliance information, unused medication information, missed medication information, patient health condition, and drug interaction information. The at least one first input comprises medication information, medication timing information, patient information, a prescription, a regimen, and a voice alert input. The at least one second input comprises collection tray operating information, collection tray timing information, disposal tray operating information, disposal tray timing information, and physiological signal.

In an embodiment, the server communicates with a drug database to extract at least one of the drug interaction information, side effects information associated with at least one medication, and composition information of the at least one medication.

In another embodiment, the user interface enables interaction between the first user, the second user, and the third user.

In yet another embodiment, the server is operable to customize the voice alert input based on the at least one first input.

In yet another embodiment, the server is operable to alert and remind at least one of the first user, the second user and the third user at a predefined time from a scheduled time.

In yet another embodiment, the server is operable to communicate with the drug database and extract a medication procedure based on the patient health condition.

In yet another embodiment, the server is operable to notify the medication compliance information, the medication non-compliance information, the unused medication information, and the missed medication information to at least one of the first user, the second user, and the third user.

In yet another embodiment, the device comprises a display that depicts at least one of the medication information, the medication timing information, the patient information, a medication procedure, and a speaker that alerts and guides the first user at a scheduled time.

In yet another embodiment, the processor is operable to depict at least one of the medication compliance information, the medication non-compliance information, the unused medication information, the missed medication information, the patient health condition, and the drug interaction information to at least one of the first user, the second user, and the third user.

In yet another aspect, a non-transitory computer storage medium is described herein. The non-transitory computer storage medium storing a sequence of instructions, which when executed by a processor, causes: provide a user interface to at least one of a first user, a second user, and a third user; receive at least one first input from at least one of the first user, the second user, and the third user via the user interface; receive at least one second input from a device; analyze the at least one first input and the at least one second input received; and estimate and record at least one of medication compliance information, medication non-compliance information, unused medication information, missed medication information, patient health condition, and drug interaction information. The at least one first input comprises medication information, medication timing information, patient information, a prescription, a regimen, and a voice alert input. The at least one second input comprises collection tray operating information, collection tray timing information, disposal tray operating information, disposal tray timing information, and physiological signal.

In yet another aspect, a method is described herein. The method comprises providing a user interface to at least one of a first user, a second user, and a third user; receiving at least one first input from at least one of the first user, the second user, and the third user via the user interface; receiving at least one second input from a device; analyzing the at least one first input and the at least one second input received; and estimating and recording at least one of medication compliance information, medication non-compliance information, unused medication information, missed medication information, patient health condition, and drug interaction information. The at least one first input comprises medication information, medication timing information, patient information, a prescription, a regimen, and a voice alert input. The at least one second input comprises collection tray operating information, collection tray timing information, disposal tray operating information, disposal tray timing information, and physiological signal.

The methods and systems disclosed herein may be implemented in any means for achieving various aspects and may be executed in a form of a non-transitory machine-readable medium embodying a set of instructions that, when executed by a machine, causes the machine to perform any of the operations disclosed herein. Other features will be apparent from the accompanying drawings and from the detailed description that follows.

### BRIEF DESCRIPTION OF THE DRAWINGS

In the present disclosure, reference is made to the accompanying drawings, which form a part hereof. In the drawings, similar symbols typically identify similar components, unless context dictates otherwise. Various embodiments described in the detailed description, and drawings, are illustrative and not meant to be limiting. Other embodiments may be used, and other changes may be made, without departing from the spirit or scope of the subject matter presented herein. It will be understood that the aspects of the present disclosure, as generally described herein, and illustrated in the Figures, can be arranged, substituted, combined, separated, and designed in a wide variety of different configurations, all of which are contemplated herein. The embodiments are illustrated by way of example and not limitation in the figures of the accompanying drawings, in which like references indicate similar elements and in which:

FIG. 1 illustrates a system view, according to one or more embodiments.

FIG. 2 illustrates an exploded view of a handheld device, according to one or more embodiments.

FIG. 3a shows a perspective view of a handheld device illustrating arrangement of a rotatable circular tray and a rotating means, according to one or more embodiments.

FIG. 3b shows a side view of a handheld device that illustrates mechanical arrangement of one or more rotatable circular trays, according to one or more embodiments.

FIGS. 3c & 3d illustrate a perspective view of a handheld device, according to one or more embodiments.

FIG. 3e-3f illustrates aligning a first compartment of a plurality of compartments with respect to an aperture, according to one or more embodiments.

FIG. 3g illustrates a bottom view depicting a first compartment aligned with respect to an aperture, according to one or more embodiments.

FIG. 3h illustrates a perspective view of a rotatable circular tray of one or more rotatable circular trays, according to one or more embodiments.

FIG. 3i illustrates a perspective view of top portion of an arm, according to one or more embodiments.

FIG. 3j illustrates a perspective view of bottom portion of an arm, according to one or more embodiments.

FIG. 3k illustrates a cap of a handheld device, according to one or more embodiments.

FIG. 3l illustrates a perspective view of a stationary circular plate, according to one or more embodiments.

FIG. 3m illustrates a sensor mountable plate, according to one or more embodiments.

FIG. 4 illustrates a top view of a rotatable circular tray of one of or more rotatable circular trays, according to one or more embodiments.

FIG. 5 illustrates a single line diagram of a handheld device comprising four rotatable circular trays, according to one or more embodiments.

FIG. 6 illustrates an architecture of a server, according to one or more embodiments.

FIG. 7a-7e illustrate a sequential user interface views, according to one or more embodiments.

FIG. 8a illustrates a fragmented front view of an outer casing of a handheld device, according to one or more embodiments.

FIG. 8b illustrates a fragmented cross section of a handheld device, according to one or more embodiments.

FIG. 8c shows a fragmented cross section of a handheld device with one or more stationary circular plates, and an outer casing, according to one or more embodiments.

FIG. 8d shows a fragmented cross section of a handheld device with one or more stationary circular plates, one or more rotatable circular trays, and an outer casing, according to one or more embodiments.

FIG. 8e illustrates a top view of a rotatable circular tray, according to one or more embodiments.

FIG. 8f is a fragmented cross section of a bottom most part of a handheld device, according to one or more embodiments.

FIG. 8g depicts a back view of a fragmented cross section of a handheld device 802 illustrating arrangement of one or more rotatable circular trays, and one or more stationary circular plates, according to one or more embodiments.

FIG. 8h shows a fragmented cross section of a rotatable circular tray illustrating arrangement of a transmitter sensor strip, and a receiver sensor strip, according to one or more embodiments.

FIG. 8i depicts a fragmented cross section illustrating arrangement of one or more rotating means and one or more arms inside a handheld device, according to one or more embodiments.

FIG. 8j depicts a handheld device without an outer casing, according to one or more embodiments.

FIG. 8k illustrates arrangement of one or more rotating means, and one or more arms, according to one or more embodiments.

FIG. 9 illustrates a block diagram of a handheld device, according to one or more embodiments.

FIG. 10 illustrates a method of tracking and ensuring medication compliance, according to one or more embodiments.

Other features of the present embodiments will be apparent from the accompanying drawings and from the detailed description that follows.

### DETAILED DESCRIPTION

Although the following detailed description contains many specifics for the purpose of illustration, a person of ordinary skill in the art will appreciate that many variations and alterations to the following details can be made and are considered to be included herein.

Accordingly, the following embodiments are set forth without any loss of generality to, and without imposing limitations upon, any claims set forth. It is also to be understood that the terminology used herein is for the purpose of describing particular embodiments only, and is not intended to be limiting. Unless defined otherwise, all technical and scientific terms used herein have the same meaning as commonly understood by one of ordinary skill in the art to which this disclosure belongs.

The articles “a” and “an” are used herein refers to one or to more than one (i.e., to at least one) of the grammatical object of the article. By way of example, “an element” means one element or more than one element.

No element, act, or instruction used herein should be construed as critical or essential unless explicitly described as such. Also, as used herein, the articles “a” and “an” are intended to include items, and may be used interchangeably with “one or more.” Furthermore, as used herein, the term “set” is intended to include items (e.g., related items, unrelated items, a combination of related items, and unrelated items, etc.), and may be used interchangeably with “one or more.” Where only one item is intended, the term “one” or similar language is used. Also, as used herein, the terms “has,” “have,” “having,” or the like are intended to be open-ended terms. Further, the phrase “based on” is intended to mean “based, at least in part, on” unless explicitly stated otherwise.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements, but may include other elements not expressly listed or inherent to such process, method, system, article, device, or apparatus.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments of the apparatus, methods, and/or articles of manufacture described herein are, for example, capable of operation in other orientations than those illustrated or otherwise described herein.

In this disclosure, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the composition's nature or characteristics would be permissible if present under the “consisting essentially of” language, even though not expressly recited in a list of items following such terminology. When using an open-ended term in this written description, like “comprising” or “including,” it is understood that direct support should also be afforded to “consisting essentially of” language as well as “consisting of” language as if stated explicitly and vice versa.

As used herein, the term “about” is used to provide flexibility to a numerical range endpoint by providing that a given value may be “a little above” or “a little below” the endpoint. However, it is to be understood that even when the term “about” is used in the present specification in connection with a specific numerical value, that support for the exact numerical value recited apart from the “about” terminology is also provided.

Reference throughout this specification to “an example”, “an instance”, “for example” means that a particular feature, structure, or characteristic described in connection with the example is included in at least one embodiment. Thus, appearances of the phrases “in an example” in various places throughout this specification are not necessarily all referring to the same embodiment.

Implementations and all of the functional operations described in this specification may be realized in digital electronic circuitry, or in computer software, firmware, or hardware, including the structures disclosed in this specification and their structural equivalents, or in combinations of one or more of them. Implementations may be realized as one or more computer program products, i.e., one or more modules of computer program instructions encoded on a computer readable medium for execution by, or to control the operation of, data processing apparatus. The computer readable medium may be a machine-readable storage device, a machine-readable storage substrate, a memory device, a composition of matter effecting a machine-readable propagated signal, or a combination of one or more of them. The term “computing system” encompasses all apparatus, devices, and machines for processing data, including by way of example a programmable processor, a computer, or multiple processors or computers. The apparatus may include, in addition to hardware, code that creates an execution environment for the computer program in question, e.g., code that constitutes processor firmware, a protocol stack, a database management system, an operating system, or a combination of one or more of them. A propagated signal is an artificially generated signal, e.g., a machine-generated electrical, optical, or electromagnetic signal that is generated to encode information for transmission to suitable receiver apparatus.

The actual specialized control hardware or software code used to implement these systems and/or methods is not limiting of the implementations. Thus, the operation and behavior of the systems and/or methods were described herein without reference to specific software code—it being understood that software and hardware can be designed to implement the systems and/or methods based on the description herein.

A computer program (also known as a program, software, software application, script, or code) may be written in any appropriate form of programming language, including compiled or interpreted languages, and it may be deployed in any appropriate form, including as a standalone program or as a module, component, subroutine, or other unit suitable for use in a computing environment. A computer program does not necessarily correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one computer or on multiple computers that are located at one site or distributed across multiple sites and interconnected by a communication network.

The processes and logic flows described in this specification may be performed by one or more programmable processors executing one or more computer programs to perform functions by operating on input data and generating output. The processes and logic flows may also be performed by, and apparatus may also be implemented as, special purpose logic circuitry, for example without limitation, a PLC (Programmable Logic Controller), an FPGA (field programmable gate array), an ASIC (application specific integrated circuit), Program-specific Standard Products (ASSPs), System-on-a-chip systems (SOCs), Complex Programmable Logic Devices (CPLDs), etc.

Processors suitable for the execution of a computer program include, by way of example, both general and special purpose microprocessors, and any one or more processors of any appropriate kind of digital computer. Generally, a processor will receive instructions and data from a read only memory or a random-access memory or both. Elements of a computer can include a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer may be embedded in another device, e.g., a mobile telephone, a personal digital assistant (PDA), a mobile audio player, a Global Positioning System (GPS) receiver, to name just a few. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory may be supplemented by, or incorporated in, special purpose logic circuitry.

To provide for interaction with a user, implementations may be realized on a computer having a display device, e.g., a CRT (cathode ray tube) or LCD (liquid crystal display) monitor, for displaying information to the user and a keyboard and a pointing device, e.g., a mouse or a trackball, by which the user may provide input to the computer. Other kinds of devices may be used to provide for interaction with a user as well; for example, feedback provided to the user may be any appropriate form of sensory feedback, e.g., visual feedback, auditory feedback, or tactile feedback; and input from the user may be received in any appropriate form, including acoustic, speech, or tactile input.

Implementations may be realized in a computing system that includes a back end component, e.g., as a data server, or that includes a middleware component, e.g., an application server, or that includes a front end component, e.g., a client computer having a graphical user interface or a Web browser through which a user may interact with an implementation, or any appropriate combination of one or more such back end, middleware, or front end components. The components of the system may be interconnected by any appropriate form or medium of digital data communication, e.g., a communication network. Examples of communication networks include a local area network (“LAN”) and a wide area network (“WAN”), e.g., the Internet.

The computing system may include clients and servers. A client and server are generally remote from each other and typically interact through a communication network. The relationship of client and server arises by virtue of computer programs running on the respective computers and having a client-server relationship to each other.

Embodiments of the present disclosure may comprise or utilize a special purpose or general-purpose computer including computer hardware. Embodiments within the scope of the present disclosure also include physical and other computer-readable media for carrying or storing computer-executable instructions and/or data structures. Such computer-readable media can be any available media that can be accessed by a general purpose or special purpose computer system. Computer-readable media that store computer-executable instructions are physical storage media. Computer-readable media that carry computer-executable instructions are transmission media. Thus, by way of example, and not limitation, embodiments of the disclosure can comprise at least two distinctly different kinds of computer-readable media: physical computer-readable storage media and transmission computer-readable media.

Computer-executable instructions comprise, for example, instructions and data which cause a general-purpose computer, special purpose computer, or special purpose processing device to perform a certain function or group of functions. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described. Rather, the described features and acts are disclosed as example forms of implementing the claims.

Physical computer-readable storage media includes RAM, ROM, EEPROM, CD-ROM or other optical disk storage (such as CDs, DVDs, etc.), magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network, such as a 5G network, or another communications connection (either hardwired, wireless, or a combination of hardwired or wireless) to a computer, the computer properly views the connection as a transmission medium. Transmissions media can include a network and/or data links which can be used to carry data or desired program code means in the form of computer-executable instructions or data structures and which can be accessed by a general purpose or special purpose computer. Combinations of the above are also included within the scope of computer-readable media.

Further, upon reaching various computer system components, program code means in the form of computer-executable instructions or data structures can be transferred automatically from transmission computer-readable media to physical computer-readable storage media (or vice versa). For example, computer-executable instructions or data structures received over a network or data link can be buffered in RAM within a network interface module (e.g., a “NIC”), and then eventually transferred to computer system RAM and/or to less volatile computer-readable physical storage media at a computer system. Thus, computer-readable physical storage media can be included in computer system components that also (or even primarily) utilize transmission media.

While this specification contains many specifics, these should not be construed as limitations on the scope of the disclosure or of what may be claimed, but rather as descriptions of features specific to particular implementations. Certain features that are described in this specification in the context of separate implementations may also be implemented in combination in a single implementation. Conversely, various features that are described in the context of a single implementation may also be implemented in multiple implementations separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products.

Even though particular combinations of features are disclosed in the specification, these combinations are not intended to limit the disclosure of possible implementations.

Further, the methods may be practiced by a computer system including one or more processors and computer-readable media such as computer memory. In particular, the computer memory may store computer-executable instructions that when executed by one or more processors cause various functions to be performed, such as the acts recited in the embodiments.

An initial overview of technology embodiments is provided below and specific technology embodiments are then described in further detail. This initial summary is intended to aid readers in understanding the technology more quickly but is not intended to identify key or essential technological features, nor is it intended to limit the scope of the claimed subject matter.

The embodiments herein and the various features and advantageous details thereof are explained more fully with reference to the non-limiting embodiments that are illustrated in the accompanying drawings and detailed in the following description.

In order to fully understand the scope of the invention, the following terms used herein are hereby defined.

As used herein “medication”, refers to a drug, a pill, a substance or any other form of medicine that is used to prevent and/or treat disease.

As used herein “medication period”, refers to a period of time that a patient has to consume prescribed medication.

As used herein “medication information” refers to information comprising medication name, medication dosage, medication composition, purpose of medication, and side effects of that medication.

As used herein “medication error” refers to any preventable event that may cause or lead to inappropriate medication use or patient harm while the medication is in the control of the health care professional, patient, caregiver, or consumer.

As used herein “patient information” refers to information comprising patient name, patient's medical history, patient's medication records, patient age, patient gender, patient ethnicity, patient's health condition, patient's prognosis report.

As used herein “regular medication”, refers to the medication that are to be consumed by the patient on a regular or daily basis.

As used herein “as-needed medication”, refers to pro re nata (PRN) medication or any other medication that are to be consumed by the patient only when needed.

As used herein “unused medication”, refers to the medication that are to be used or left or remaining in the device.

As used herein “drug interaction”, is defined as an interaction between a drug and another substance that prevents the drug from performing as expected. The drug interaction may increase or decrease the effectiveness of the drugs or their side effects.

As used herein “patient health condition”, refers to a medical condition or status of the patient at a particular timing. The patient health condition may assist in detecting the likelihood of prone to a disease or severity (e.g. progression, regression) of the disease.

As used herein “medication compliance”, is defined as the extent to which a patient correctly follows medical advice (e.g., taking medication, treatment regimen, lifestyle tips, advice concerning disease management).

As used herein “medication non-compliance” is commonly used in regard to a patient who does not take a prescribed medication or follow a prescribed course of treatment.

As used herein “medication compliance information”, refers to information comprising medication consumption timing, medication information, and patient information.

As used herein “unused medication information”, refers to information comprising amount and details of medication that are left or remaining or unused in a device and corresponding medication information.

As used herein “missed medication information”, refers to information comprising amount and details of medication that are missed or non-consumed by a patient and corresponding medication information.

As used herein “collection tray” refers to a collection point where a patient or a user can collection dispensed substance. The collection tray referred herein is also a secured tray where only an intended user (e.g. a patient, a caregiver, etc.) is authorized to collect dispensed medication.

As used herein “disposal tray” refers to a collection and storage container that comprises the medication that are missed by the patient and the caregiver during medication period. The disposal tray referred herein is also a secured tray where only an intended user (e.g. a pharmacist, a caregiver, etc.) is authorized to collect missed medication.

As used herein “as-needed medication tray” refers to a storage container that holds pro re nata (PRN) medication i.e. as-needed medication.

In an embodiment, the system comprises a device and a server communicatively coupled to a computing unit.

In another embodiment, the system is operable to dispense at least one medication at a scheduled time.

In yet another embodiment, the system is operable to alert or remind at least one of a patient, a caregiver, and a nurse at a predefined time from a scheduled time to consume the medication.

In yet another embodiment, the system is operable to monitor and track medication compliance, medication non-compliance, unused medication, missed medication, drug interaction, etc.

In yet another embodiment, the system automatically prevents medication error by dispensing exact dosage of the medication at a scheduled time and diverting the medication that are missed by the patient to a disposal tray.

In yet another embodiment, the system is operable to notify medication compliance, medication non-compliance, unused medication, missed medication, drug interaction, etc. to at least one of a patient, a caregiver, a pharmacist, a nurse, an insurance company, and a doctor.

In yet another embodiment, the system is operable to notify medication compliance, medication non-compliance, unused medication, missed medication, drug interaction, etc. to at least one of a patient, a caregiver, a pharmacist, a nurse, an insurance company, and a doctor.

In yet another embodiment, the system is operable to divert the missed medication from a collection tray to a disposal tray after a predefined time from the scheduled time.

In yet another embodiment, the system enables or authorizes only an intended user (e.g. the pharmacist and the caregiver) to access a disposal tray. In other words, the system restricts the patient from accessing the disposal tray thereby preventing the patient from taking double dose, overdose, etc.

In yet another embodiment, the system enables or authorizes only an intended person (e.g. the patient and the caregiver) to access a collection tray and collect the medication. In other words, the system restricts non-intended persons (e.g. kids, guests, etc.) from accessing the collection tray thereby tracking and ensuring medication compliance.

In yet another embodiment, the system is operable to operate an as-needed medication tray via a user interface provided to at least one of the patient, the caregiver, the nurse, the pharmacist, the insurance company and the doctor.

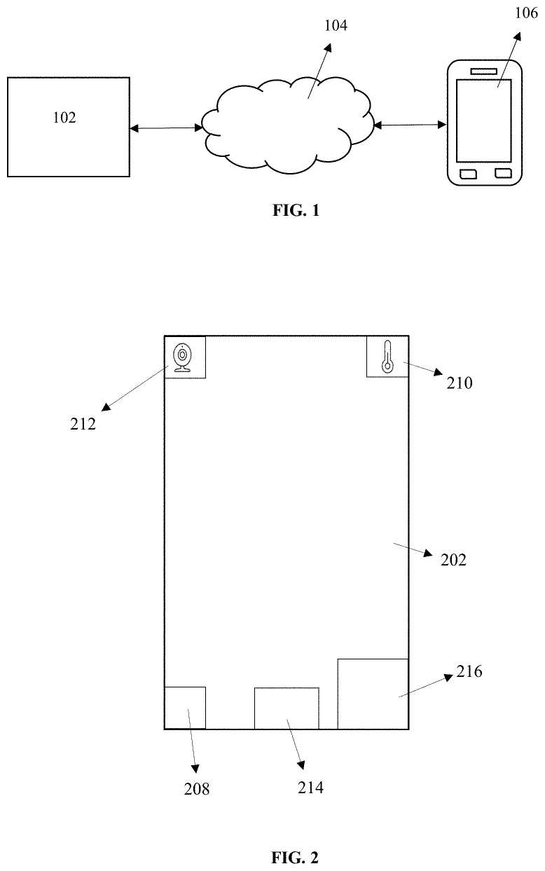

FIG. 1 illustrates a system view, according to one or more embodiments. The system comprises a handheld device 102, a server 104, and a computing unit 106. In an embodiment, the handheld device 102 described herein is adapted to dispense medication on a daily or regular basis. In another embodiment, the handheld device 102 described herein is adapted to dispense at least one substance on a daily and or a regular basis. In yet another embodiment, the handheld device 102 comprises an as-needed medication tray that is adapted to operate and dispense as-needed medication only when needed. The handheld device 102 is communicatively coupled with the server 104 and the computing unit 106 via a wireless communication network to operate the handheld device 102 remotely. In an embodiment, the handheld device 102 is communicatively coupled with the computing unit 106 via a near field communication network (e.g. Bluetooth®, Wi-Fi®, Zigbee®, etc.) The handheld device 102 is a portable device. The handheld device 102 may be a rechargeable and battery powered device. The handheld device 102 may be a direct plug-in device (i.e. directly connected to an electric supply). In an embodiment, the handheld device 102 is adapted to hold and dispense the medication for multiple patients at one or more scheduled times.

The computing unit 106 may be a personal digital assistant. In an embodiment, the computing unit 106 comprises at least one of a smart phone, a computer, a tablet etc. In another embodiment, the system comprises the more than one computing unit 106 (i.e. a computing unit for a first user, a second user, and a third user). The first user may be a patient. The second user may be a caregiver, a nurse etc. The third user may be a pharmacist, a doctor, an insurance company, etc. The server 104 monitors and co-ordinates one or more activities and functions performed by the handheld device 102 and the computing unit 106. The server 104 further keep tracks of the one or more activities and functions performed by the handheld device 102 and the computing unit 106 to estimate and record at least one of medication compliance information, medication non-compliance information, unused medication information, missed medication information, patient health condition, and drug interaction information.

FIG. 2 illustrates an exploded view of a handheld device 202, according to one or more embodiments. The handheld device 202 comprises an identification sensor 208, a physiological sensor 210, a camera system 212, a collection tray 214, and an input/output (I/O) unit 216. The identification sensor 208 may be one of a radio-frequency identification (RFID) sensor, a biometric sensor, and a fingerprint sensor. The identification sensor 208 may be affixed or associated with a collection tray 214. In an embodiment, the identification sensor 208 may be placed adjacent to the collection tray 214. The identification sensor 208 is configured to recognize identification data associated with a first user (e.g. a patient, a caregiver, etc.) and communicate a signal to the collection tray 214 in response to the determination of the identification data associated with the first user.

The collection tray 214 upon receiving the signal operates and enables the first user to collect at least one medication from the collection tray 214, when the identification data is recognized by the identification sensor 208. For an instance, when the first user tries to access the collection tray 214, the identification sensor 208 enables the first user (e.g. patient, caregiver, etc.) to collect the at least one medication dispensed from the collection tray 214. In an embodiment, the collection tray restricts accessibility, when the identification sensor 208 do not recognizes the identification data. For an instance, when a third person (e.g. kids, guest at home, etc.)tries to access the collection tray 214, the identification sensor associated with the collection tray 214 restricts the access and therefore, the at least one medication dispensed from the plurality of compartments still remains secured within the collection tray 214 of the handheld device 202 for a first predefined period of time before and after a scheduled time. In an embodiment, the first predefined period of time ranges from ten to twenty minutes. In another embodiment, the first predefined period of time is customized by at least one of the first user, the second user, and a third user via a user interface provided.

The physiological sensor 210 is affixed to the handheld device 202. In an embodiment, the handheld device 202 is positioned at top-right corner of the handheld device 202. The physiological sensor 210 is configured to acquire at least one physiological signal from the first user (e.g. the patient). In an embodiment, the physiological sensor 210 may be at least one of a temperature sensor, a blood pressure sensor, an electrocardiogram (ECG) electrode, etc. In yet another embodiment, the physiological sensor 210 is operated by a second user (e.g. caregiver, pharmacist, doctor, etc.) remotely via the user interface provided through a computing unit. In yet another embodiment, the physiological sensor 210 is operated by the first user (e.g. the patient, the caregiver etc.) physically.

The camera system 212 is configured to capture at least one of an image, and a video of a scene around the handheld device 202 for a second predefined period of time before and after the scheduled time. In an embodiment, the second predefined period of time ranges from ten to twenty minutes. In another embodiment, the second predefined period of time is customized by at least one of the first user, the second user, and the third user via the user interface provided. In yet another embodiment, the camera system 212 comprises an artificial intelligence enabled image processing unit that enables the camera system 212 to analyze at least one of the image, and video captured to determine whether the patient is accessing the collection tray 214 and consuming the medication. In yet another embodiment, the camera system 212 comprises an artificial intelligence enabled voice processing unit that enables to analyze at least one voice input received from the first user and to determine whether the patient is accessing the collection tray 214, collecting and consuming the medication. In yet another embodiment, the camera system 212 is adapted to ensure medication compliance. In another embodiment, the camera system 212 is configured to determine medication compliance, medication non-compliance, unused medication, missed medication, drug interaction, etc.

The input/output (I/O) unit 216 comprises at least one of a speaker, a microphone and a display. The display receives a signal from a server and the handheld device 202 and depicts at least one of medication information, patient information, drug interaction information, unused medication information, missed medication information, etc. The display is also configured to depict a medication procedure that guides the patient how to consume the medication. The speaker provides an audio output. The audio output may be at least one of a customized voice alert, a reminder, etc. The microphone is configured to acquire voice input, which may assist during interaction between the patient, the doctor, the nurse, the pharmacist, the doctor and the insurance company.

In an embodiment, the handheld device 202 comprises a secondary medication container that is to be utilized by the first user (e.g. the patient) when the first user is in travel or out of station. The secondary medication container is assembled or disassembled from the handheld device 202. In an embodiment, the secondary medication container is an open and close container. In another embodiment, the secondary medication container may be rechargeable and battery powered. In yet another embodiment, the secondary medication container enables to input an access code to operate and dispense the medication. The first user is enabled to request the access code via a user interface provided through a computing unit. The secondary medication container operates and dispenses the medication when the access code is recognized or matched. In an embodiment, the handheld device 202 enables to input the access code to assemble or disassemble the secondary medication container from the handheld device 202. In another embodiment, the handheld device 202 ejects or disassembles the secondary medication container from the handheld device 202 when the identification data associated with the first user is recognized by a second identification sensor associated with the secondary medication container.

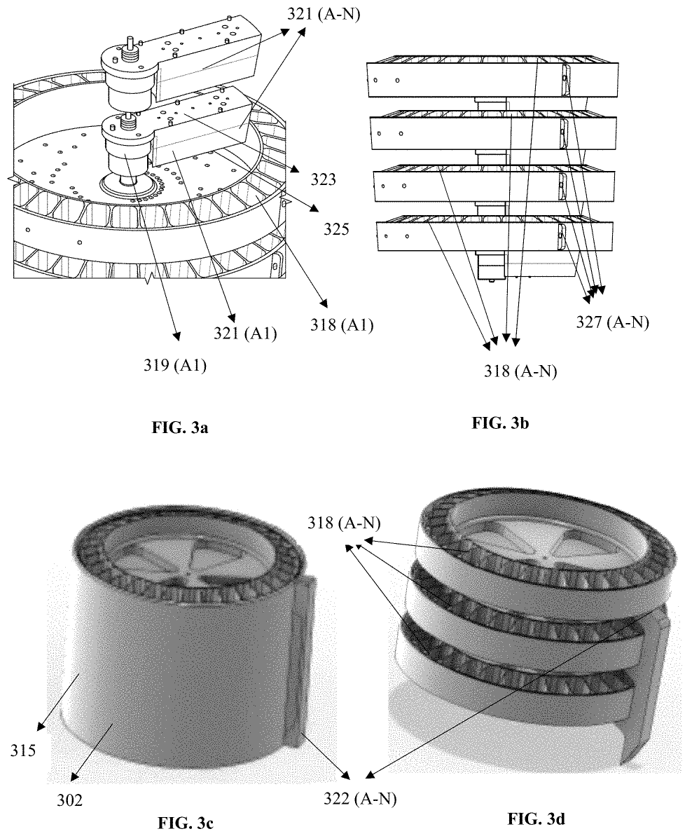

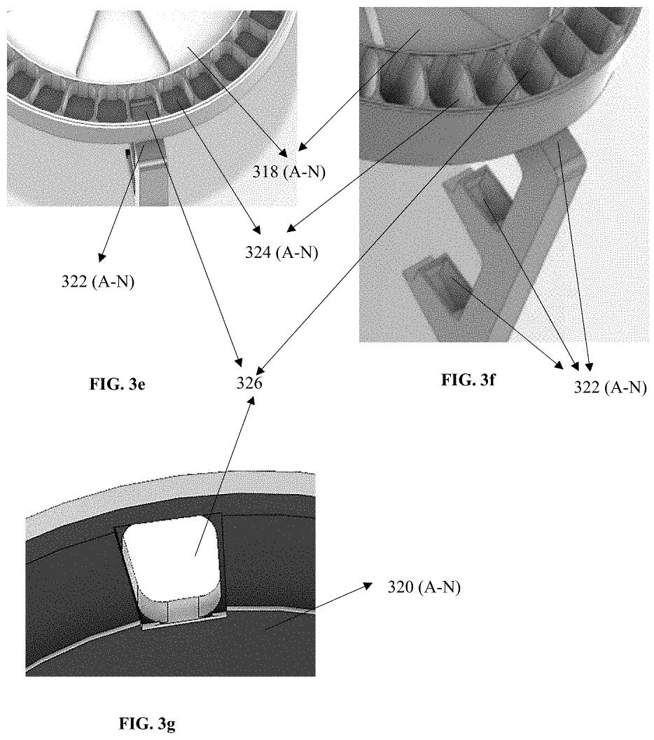

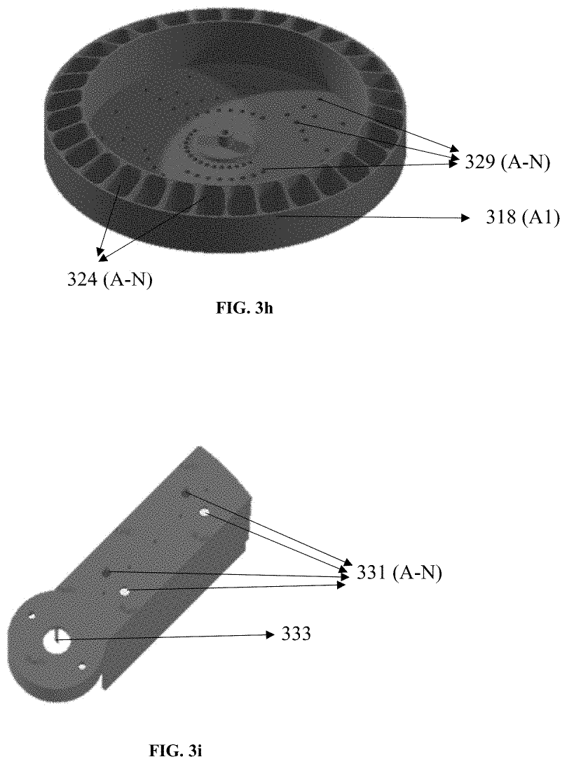

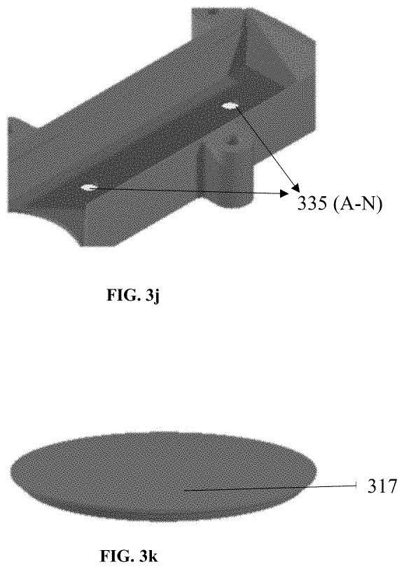

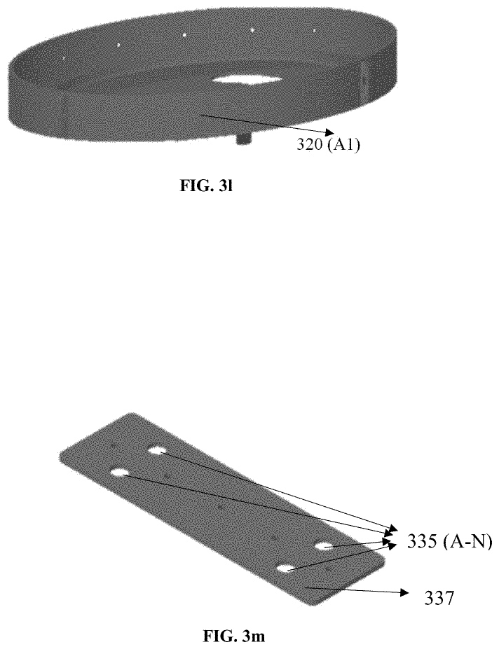

FIG. 3a shows a perspective view of a handheld device illustrating arrangement of a rotatable circular tray 318 (Al) and a rotating means 319 (Al), according to one or more embodiments. The rotatable circular tray 318 (Al) is coupled to the rotating means 319 (Al) via an arm 321 (Al). The arm 321 (Al) comprises a top plate 323 and a bottom plate 325. The top plate 323 holds a transmitter sensor trip for a previous rotatable circular tray and the bottom plate 35 holds a receiver sensor strip for the rotatable circular tray 318 (Al) (i.e. current rotatable circular tray). The arm 321 Al guides the rotatable circular tray 318 (Al) as per the actuation or rotation of the rotating means to align at least one compartment of a plurality of compartments with respect to an aperture of a stationary circular plate.

FIG. 3b shows a side view of a handheld device 302 that illustrates mechanical arrangement of one or more rotatable circular trays 318 (A-N), according to one or more embodiments. The side view further shows one or more handles 327 (A-N) that are adapted to pull and release the one or more rotatable circular trays from the handheld device while refilling medication into the handheld device 302.

FIGS. 3c & 3d illustrate a perspective view of a handheld device 302, according to one or more embodiments. FIG. 3c shows the handheld device 302 with an outer casing while FIG. 3d shows the handheld device 302 without the outer casing. The handheld device 302 comprises one or more rotatable circular trays 318 (A-N), one or more stationary circular plates 320 (A-N), and one or more channels 322 (A-N). A rotatable circular tray 318 (A-N) of the one or more rotatable circular trays 318 (A-N) comprises a plurality of compartments 324 (A-N). The plurality of compartments 324 (A-N) may comprise regular medication. In an embodiment, a compartment of the plurality of compartments 324 (A-N) comprises at least one medication. In another embodiment, the compartment of the plurality of compartments 324 (A-N) comprises more than one medication. In yet another embodiment, the rotatable circular tray 318 (A-N) comprises thirty-one medication compartments (i.e. a compartment holds medication for a day of a month) and an empty compartment. Each stationary circular plate of the one or more stationary circular plates comprises an aperture 326 (A-N). The empty compartment is adapted to be aligned with the aperture in the stationary circular plate while filling the medication or loading the one or more rotatable circular trays 318 (A-N) in the handheld device 302. For a first instance, the handheld device 302 comprises four rotatable circular tray 318 (A-N) and four stationary circular plates 320 (A-N), for holding the medication for morning, noon, evening and night. For a second instance, the handheld device 302 may comprise five rotatable circular trays 318 (A-N) and five stationary circular plates 320 (A-N), for holding the medication for before breakfast, after breakfast, noon, evening and night (i.e. the count of the one or more rotatable circular trays 318 (A-N) and the one or more stationary circular plates 320 (A-N) can extended or reduced in the handheld device 302 as per medication regimen of the patient). For a third instance, the handheld device 302 may comprise eight rotatable circular trays 318 (A-N) and eight stationary circular plates 320 (A-N), for holding the medication of two patients (i.e. multiple patients) individually for morning, noon, evening and night in the handheld device 302.

The one or more stationary circular plates 320 (A-N) are mechanically positioned below respective circular trays of the one or more rotatable circular trays 318 (A-N). The one or more stationary circular plates 320 (A-N) are adapted to hold the one or more rotatable circular trays 318 (A-N) in position while the handheld device 302 is operating. In an embodiment, the one or more stationary circular plates 320 (A-N) are adapted to hold the medication within its respective compartment of the plurality of compartments 324 (A-N). In another embodiment, the one or more stationary circular plates 320 (A-N) are fastened below respective circular trays of the one or more rotatable circular trays 318 (A-N) to stop spillage of the medication and mixing of the medication between the plurality of compartments 324 (A-N) and between the one or more rotatable circular trays 318 (A-N). In yet another embodiment, the plurality of compartments 324 (A-N) is also designed in such a way to stop the spillage of the medication and mixing of the medication between the plurality of compartments 324 (A-N). In yet another embodiment, the medication in the one or more rotatable circular trays 318 (A-N) are preloaded or filled by at least one of the pharmacist, the nurse, and the caregiver.

The one or more channels 322 (A-N) are positioned below one or more apertures 326 (A-N). The one or more channels 322 (A-N) guide the medication dispensed from the plurality of compartments 324 (A-N) to a collection tray. The one or more channels 322 (A-N) are designed in such a way that the medication dispensed from one rotatable circular tray 318 (A-N) do not arrive at the other rotatable circular trays 318 (A-N) or mix up with the other medication in the other rotatable circular trays 318 (A-N). In an embodiment, the one or more channels 322 (A-N) operates without any external energy and guides the dispensed medication to the collection tray. In another embodiment, the one or more channels 322 (A-N) utilizes gravitational force as energy source to guide the dispensed medication to the collection tray.

In an embodiment, the handheld device 302 comprises an as-needed medication tray and a disposal tray. The as-needed medication tray holds as-needed medication that may be needed for the patient only on demand. The as-needed medication tray is filled by at least one of a pharmacist, a nurse, a caregiver, and a doctor. In another embodiment, the as-needed medication tray is pre-loaded by at least one of the pharmacist, the nurse, the caregiver, and the doctor. The as-needed medication tray is operable via a user interface by at least one of a first user, a second user, and a third user when needed. The as-needed medication tray also designed in such a way that there is no spillage or any mix up of the medication with other regular medication in the rotatable circular tray. In an embodiment, the as-needed medication tray dispenses the as-needed medication upon receiving an input from a server, a computing unit, etc. In another embodiment, the as-needed medication tray also dispenses the as-needed medication based on information extracted from a drug database and health condition of the patient to restrict the patient from taking double dose, overdose, etc. In yet another embodiment, the handheld device diverts the as-needed medication, that are dispensed but missed, to the disposal tray.

The collection tray holds the dispensed medication for a first predefined period of time. The first predefined period of time may range from 10 minutes to 20 minutes. In an embodiment, the predefined period of time is customized and altered through the user interface provided. The collection tray secures the medication until the intended user (e.g. the patient, the caregiver, the nurse, etc.) accesses the collection tray and collects the medication. The collection tray operates and allows the intended user to collect the medication only when identification data associated with the intended user is recognized and matched.

The disposal tray is positioned below the collection tray. The medication that are arrived at the collection tray but missed (i.e. missed medication) are diverted or dropped into the disposal tray. In an embodiment, the disposal tray comprises an identification sensor that recognizes identification data associated with at least one of a caregiver, a pharmacist, a doctor, and an insurance company to provide access and dispose the missed medication. The identification sensor affixed in the disposal tray is configured not to provide authorization to the patient to access the disposal tray and collect the missed medication. The disposal tray secures and holds the missed medication to restrict the patient from taking double dose, overdose, etc.

The handheld device 302 further comprises a microcontroller that is configured to provide excitation and coordinate with the server. In an embodiment, the handheld device 302 comprises one or more rotating means. The one or more rotating means comprise one of (a) one or more permanent magnet direct current (PMDC) motor, (b) one or more stepper motors, and (c) one or more servo motors. In an embodiment, the one or more rotating means comprise one or more shunt direct current (DC) motors. The field is connected in parallel (shunt) with the armature windings in the one or more shunt DC motors. The one or more shunt DC motors provide great speed regulation due to the fact that the shunt field can be excited separately from the armature windings, which also offers simplified reversing controls. The one or more rotating means are mechanically coupled to the one or more rotatable circular trays 318 (A-N) via one or more arms. The one or more rotating means actuate the one or more rotatable circular trays 318 (A-N) and align a first compartment of the plurality of compartments 324 (A-N) with respect to the aperture in the stationary circular plate at a first scheduled time on a first day of medication period. In an embodiment, the one or more rotating means actuate the one or more rotatable circular trays 318 (A-N) to sequentially (e.g. clockwise, anticlockwise) rotate and align the plurality of compartments 324 (A-N) with respect to the apertures at respective scheduled times on respective days of medication to empty the plurality of compartments and dispense the medication. In another embodiment, a rotatable circular tray of the one or more rotatable circular trays 318 (A-N) is mechanically coupled to a rotating means to actuate and align with respect to the aperture.

The first compartment holds the medication for a first day of medication. A second compartment holds the medication for a second day of medication. Similarly, successive compartments hold the medication for successive days of medication for a medication period. A first rotatable circular tray of the one or more rotatable circular trays 318 (A-N) holds the medication for a first scheduled time (e.g. morning). A second rotatable circular tray of the one or more rotatable circular trays 318 (A-N) holds the medication for a second scheduled time (e.g. noon). Therefore, the first rotatable circular tray dispenses the medication from the first compartment at the first scheduled time on the first day of medication when the first compartment is aligned with respect to the aperture. The first rotatable circular tray dispenses the medication from the second compartment at the first scheduled time on the second day of medication when the second compartment is aligned with respect to the aperture. Similarly, the second rotatable circular tray dispenses the medication from its first compartment at the second scheduled time on the first day of medication. Further, the second rotatable circular tray dispenses the medication from its second compartment at the second scheduled time on the second day of medication. The plurality of compartments comprises surface area equal to surface area of the aperture (i.e. each compartment of the plurality of compartments fits exactly with the aperture) so that when the plurality of compartments 324 (A-N) aligns with the aperture, the plurality of compartments 324 (A-N) dispenses or drops the medication completely via the aperture to the one or more channels 322 (A-N).

In another embodiment, the handheld device 302 comprises the one or more rotating means as one or more servo motors and a plurality of fourth sensors. The plurality of fourth sensors are positioned parallel with respect to the plurality of compartments 324 (A-N) to provide feedback to the one or more servo motors and align the first compartment of the first rotatable circular tray with respect to the aperture at the first scheduled time on the first day of medication. Similarly, the one or more servo motors actuate and align the second compartment of the first rotatable circular tray with respect to the aperture at the first scheduled time on the second day of medication. The plurality of compartments 324 (A-N) dispenses or drops the medication when the plurality of compartments 324 (A-N) are aligned with respect to the aperture.

FIG. 3e-3f illustrates aligning a first compartment of a plurality of compartments 324 (A-N) with respect to an aperture 326, according to one or more embodiments. The first compartment of a rotatable circular tray 318 (A-N) is exactly aligned with respect to the aperture 326 at a first scheduled time on a first day of medication period. Similarly, a second compartment of the rotatable circular tray 318 (A-N) is exactly aligned with respect to the aperture 326 at a first scheduled time on a second day of medication period. Similarly, successive compartments of the plurality of compartments 324 (A-N) are exactly aligned with respect to the aperture 326 on successive days of the medication period to dispense the medication.

FIG. 3g illustrates a bottom view depicting a first compartment aligned with respect to an aperture 326, according to one or more embodiments. The first compartment of a rotatable circular tray 318 (A-N) is aligned with respect to the aperture 326 at a first scheduled time on a first day of medication period. FIG. 3g further depicts that the aperture 326 comprises surface area equal to surface area of at least one compartment of a plurality of compartments 324 (A-N). FIG. 3g further depicts that the aperture 326 comprises shape equal to shape of the plurality of compartments 324 (A-N). The plurality of compartments 324 (A-N) comprises the shape and the surface area equal to the shape and the surface area of the aperture 326 so that when the plurality of compartments 324 (A-N) and the aperture 326 are aligned, the medication in the plurality of compartments 324 (A-N) is completely dropped or dispensed.

FIG. 3h illustrates a perspective view of a rotatable circular tray 318 (Al) of one or more rotatable circular trays 318 (A-N), according to one or more embodiments. The perspective view of the rotatable circular tray 318 (Al) shows an array of identifiers 329 (A-N) arranged in one or more patterns. The one or more identifiers 329 (A-N) may be one or more holes, braille script etc. FIG. 3h also shows a plurality of compartments 324 (A-N) that are adapted to hold medication. Out of the plurality of compartments 324 (A-N), one compartment is empty.

FIG. 3i illustrates a perspective view of top portion of an arm 321, according to one or more embodiments. The perspective view of the top portion of the arm 321 shows markings 331 (A-N), where a transmitter sensor strip or a receiver sensor strip is to be fixed. The top portion of the arm 321 shows a mounting portion 333. The mounting portion 333 is adapted to mount the arm 321 onto a rotating means.

FIG. 3j illustrates a perspective view of bottom portion of an arm 321, according to one or more embodiments. The bottom portion of the arm 321 shows markings 335 (A-N), where a transmitter sensor strip or a receiver sensor strip is to be fixed.

FIG. 3k illustrates a cap 317 of a handheld device, according to one or more embodiments. A rotatable circular tray of the handheld device comprises the cap 317 affixed onto its top to secure medication in position while the handheld device is operating. The cap 317 not only holds the medication in position, but also stops the spillage of the medication when the handheld device is flipped. Further the cap 317 is also adapted to prevent mixing up of the medication between a plurality of compartments and between one or more rotatable circular trays. In an embodiment the cap 317 secures the medication in its position to prevent spillage or mixing of the medication thereby, preventing dispense of wrong medication.

FIG. 3l illustrates a perspective view of a stationary circular plate 320 (Al), according to one or more embodiments. The stationary circular plate 320 (Al) is adapted to hold a rotatable circular tray in its position while the rotatable circular tray is operating.

FIG. 3m illustrates a sensor mountable plate 337, according to one or more embodiments. The sensor mountable plate 337 is attached onto of a top portion of an arm. The sensor mountable plate 337 is adapted to hold or mount one of a transmitter sensor strip and a receiver sensor strip. The sensor mountable plate 337 shows markings 335 (A-N), where one of the transmitter sensor strip, and the receiver sensor strip is to be fixed.

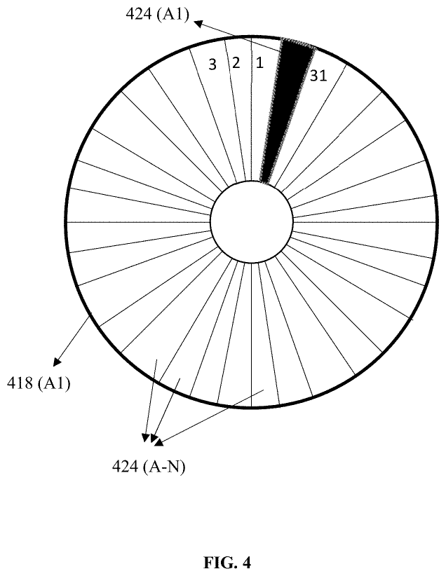

FIG. 4 illustrates a top view of a rotatable circular tray 418 (Al) of one of or more rotatable circular trays 418 (A-N), according to one or more embodiments. The rotatable circular tray 418 (Al) comprises a plurality of compartments 424 (A-N). A compartment of the plurality of compartments 424 (A-N) holds medication for a day of a medication period. The plurality of compartments 424 (A-N) may hold regular medication. In an embodiment, the rotatable circular tray 418 (A-N) comprises thirty-one medication compartments (i.e. a compartment holds medication for a day of a month) and an empty compartment 424 (Al). The empty compartment 424 (Al) is aligned with respect to an aperture once the medication is filled or loaded in the plurality of compartments 424 (A-N). In another embodiment, count of the plurality of compartments is customized and altered as per the medication period. For a first instance, the rotatable circular tray 418 (Al) comprises twenty-eight medication compartments (i.e. a compartment holds medication for a day of a February month) and an empty compartment. For a second instance, the rotatable circular tray 418 (Al) comprises twenty-nine medication compartments (i.e. a compartment holds medication for a day of a February month in a leap year) and an empty compartment. In yet another embodiment, the rotatable circular tray 418 (Al) comprises thirty-one medication compartments out of which only the first twenty-eight compartments or first twenty-nine compartments of the rotatable circular tray are filled with the medication.

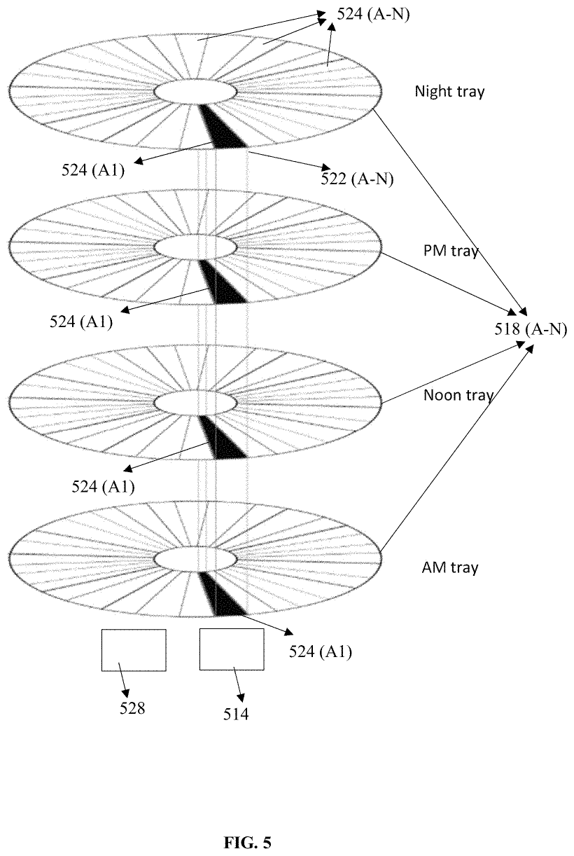

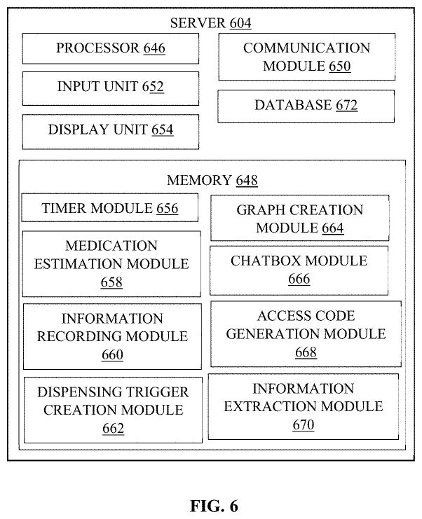

FIG. 5 illustrates a single line diagram of a handheld device 502 comprising four rotatable circular trays 504 (A-N), according to one or more embodiments. A rotatable circular tray 504 (A-N) comprises a plurality of compartments 524 (A-N). The plurality of compartments 524 (A-N) comprise thirty-one compartments that holds medication and an empty compartment 524 Al. The empty compartment 524 Al of the four rotatable circular trays 504 A-N), are aligned with respect to the aperture initially, once the medication is loaded into the plurality of compartments 524 (A-N). The four rotatable circular trays 504 (A-N) holds the medication for different medication timings (e.g. AM, noon, PM, night). The one or more channels 522 (A-N) guides the medication dispensed from the plurality of compartments 524 (A-N) to a collection tray 514. The collection tray 514 secures and holds the medication for a predefined period of time until a patient or a caregiver collects the medication. The handheld device 502 further comprises a disposal tray 528 positioned adjacent or below the collection tray 514. The disposal tray 528 collects and secures the medication from the collection tray 514 that are missed by the patient.

FIG. 6 illustrates an architecture of a server 604, according to one or more embodiments. The server 604 comprises a processor 646, a memory 648, a communication module 650, an input unit 652, a display 654, and a database 672. The memory 648 further comprises a timer module 656, a medication estimation module 658, an information recording module 660, a dispensing trigger creation module 662, a graph creation module 664, a chat box module 666, an access code generation module 668, an information extraction module 670, and a database 672. The input unit 652 is configured to receive one or more inputs from at least one of a first user, a second user, and a third user. The one or more inputs comprise at least one first input, and at least one second input. The at least one first input comprises medication information, medication timing information, patient information, a prescription, a regimen and a voice alert input. The at least one second input comprises collection tray operating information, collection tray timing information, disposal tray operating information, disposal tray timing information, and physiological signal. The communication module 650 obtains at least one signal and data from the handheld device 602 and communicates information in response. The processor 646 is configured to execute one or more instructions stored in the above-mentioned modules. The timer module 656 is configured to realize present time and keep track of the scheduled time. The timer module 656 is further configured to notify and alert medication compliance, medication non-compliance, unused medication, missed medication, drug interaction at a fourth predefined time from the scheduled time to at least one of the first user, the second user, and the third user. The medication estimation module 658 analyzes the at least one first input and the at least one second input. The medication estimation module 658 then estimates at least one of medication compliance information, medication non-compliance information, unused medication information, missed medication information, as-needed medication information, drug interaction information etc.

The information recording module 660 records the estimated information for at least one patient. The dispensing trigger creation module 662 creates a dispensing trigger signal for the at least one patient at the scheduled time based on the at least one first input (e.g. data such as regimen) and the at least one second input (e.g. signal such as sensor signal). The dispensing trigger signal is communicated to the handheld device 602 to dispense the medication from the plurality of compartments at the scheduled time. The graph creation module 664 creates charts, graphs (e.g. unused medication charts, missed medication charts, etc.) of the medication of at least one patient upon receiving the at least one first input. The chat box module 666 enables interaction between the first user, the second user, and the third user. The chat box module 666 is configured to schedule a video call between the first user, the second user, and the third user to minimize in-person meetings. The video call enables the doctor to safely treat the patient and prescribe medication, intervention in scenarios like contagious diseases (e.g. COVID 19). The chat box module 666 is further configured to enable the first user (e.g. patient) to book an appointment with the third user (e.g. doctor).

The access code generation module 668 is configured to request an access code from the third user (e.g. pharmacist, doctor, nurse, etc.) by the first user (e.g. the patient) for a secondary medication container. The secondary medication container is enclosed within the handheld device 602. The secondary medication container is provided to the first user to be utilized when the first user is in travel or out of station. The secondary medication container allows the first user to access and collect the medication when the first user provides the access code. The information recording module 660 tracks and records the access code to ensure the medication compliance, track dosage information of the as-needed medication, etc. even when the patient travels or out of station. The information recording module 660 further tracks and records as-needed medication information, unused medication information, missed medication information, etc. The information recording module 660 further tracks and records patient information, medical history of the patient, health condition, etc.

The information extraction module 670 is configured to extract information from an external database (e.g. drug database, clinical database, etc.) and render via at least one of the user interface to the computing unit, and the display 654 in the handheld device 602. The information extracted may comprise at least one of side effects, drug interaction information, purpose of the medication, dosage of the medication for the patient, prognosis etc. In one embodiment, the information extraction module 670 automatically extracts the information from the external database at the scheduled time. In another embodiment, the information extraction module 670 extracts the information from the database upon receiving the one or more inputs from at least one of the first user, the second user, and the third user. The information recorded and the information extracted are depicted to at least one of the first user, the second user, and, the third user. The database 672 stores medication information, patient information, side-effects of the medication, medication dosage, etc. to readily depict to the user when needed.

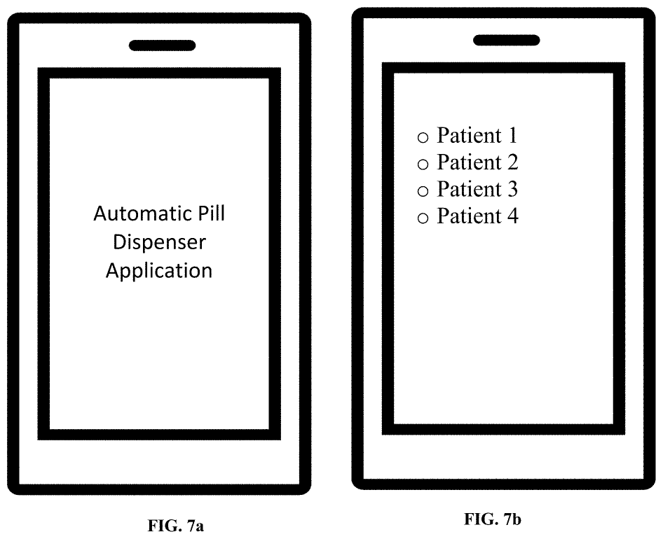

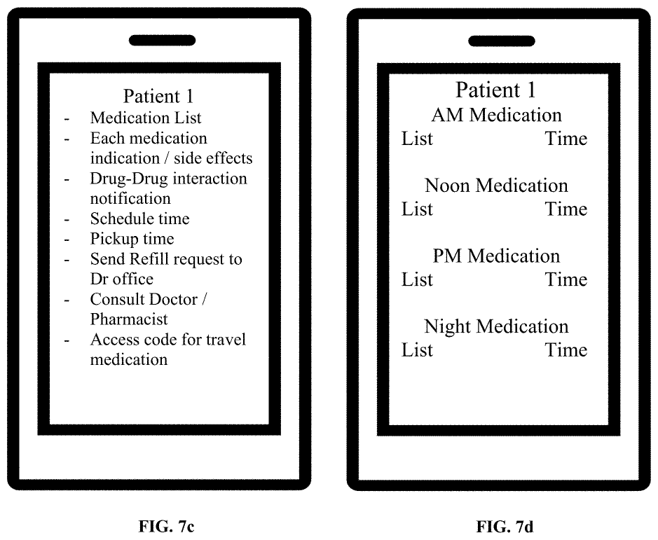

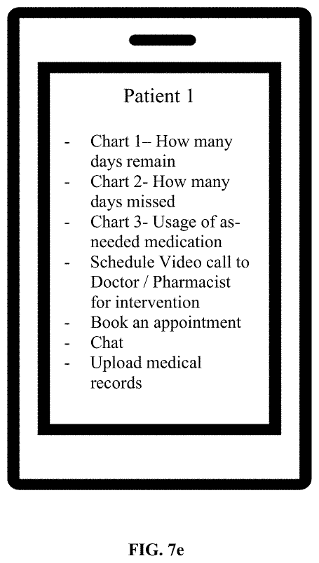

FIG. 7a-7e illustrate a sequential user interface views, according to one or more embodiments. FIG. 7a depicts a first user interface view provided to at least one of a first user, a second user, and a third user. The first user may be a patient. The second user may be at least one of a caregiver, and a nurse. The third user may be at least one of a pharmacist, a doctor and an insurance company. FIG. 7b illustrates a second user interface view depicting a list of patients for which medications are loaded or filled into a handheld device. The second user interface view further enables at least one of the first user, the second user, and the third user to at least one of add, edit, correct, and remove a patient from the list of patients depicted.

FIG. 7c illustrates a third user interface view depicting a list of action items for a first patient in the list of patients to at least one of the first user, the second user, and the third user. The third user interface view depicts the list of action items upon clicking the first patient from the list of patients shown in the second user interface view. The list of action items of the first patient comprises a medication list, medication indication/side effects, drug interaction notification, schedule time, pickup time, send refill request to doctor or pharmacist, consult doctor or pharmacist, access code for travel medication, etc. The list of actions items may at least one of receive and provide information. Action items such as the medication list, Schedule time, Pickup time, etc. may receive information from at least one of the first user, the second user, and the third user. The action items such as medication indication/side effects, etc. may extract information from a drug database and renders the information to at least one of the first user, the second user, and the third user.

FIG. 7d illustrates a fourth user interface view depicted upon clicking the action item “medication list”. The fourth user interface view enables at least one of the first user, the second user, and the third user to provide medication information such as medication name, medication dosage, medication timing, etc. FIG. 7e illustrates a fifth user interface view depicted upon clicking the action item “Consult Doctor/Pharmacist”. The fifth user interface view enables at least one of the first user, the second user, and the third user to create missed medication charts, unused medication charts, schedule video call with doctor, book an appointment, chat, upload medical records, view usage of as-needed medication etc. to enhance consultation or interaction. For an instance, the user upon clicking “Chart 1” action item, a server extracts information and creates a chart and communicates to a computing unit. Similarly, for other action items, the communication is established between the server and the computing unit to render an output.

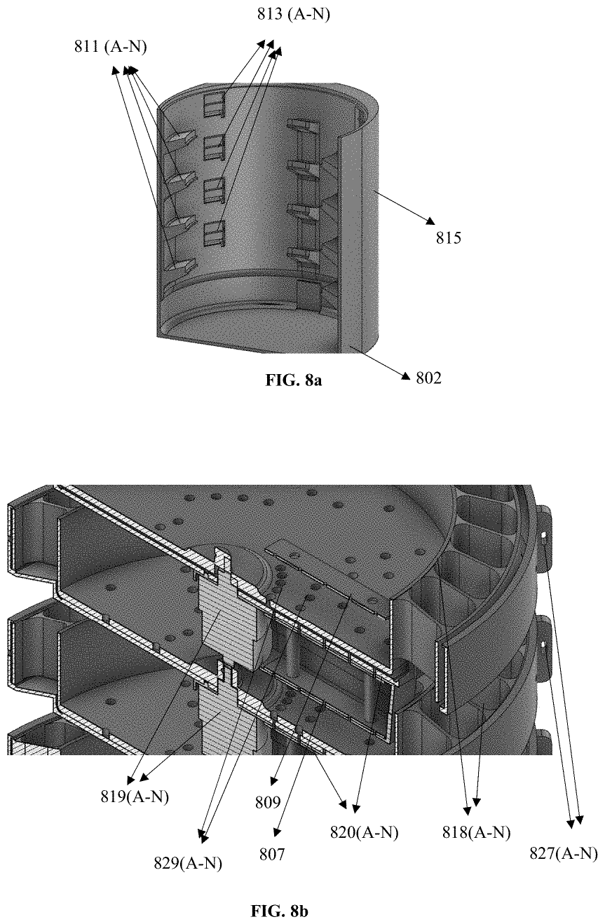

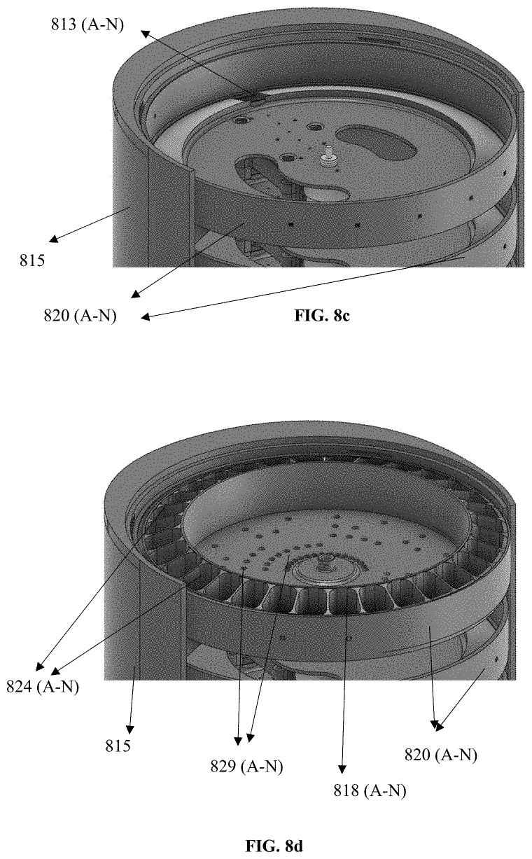

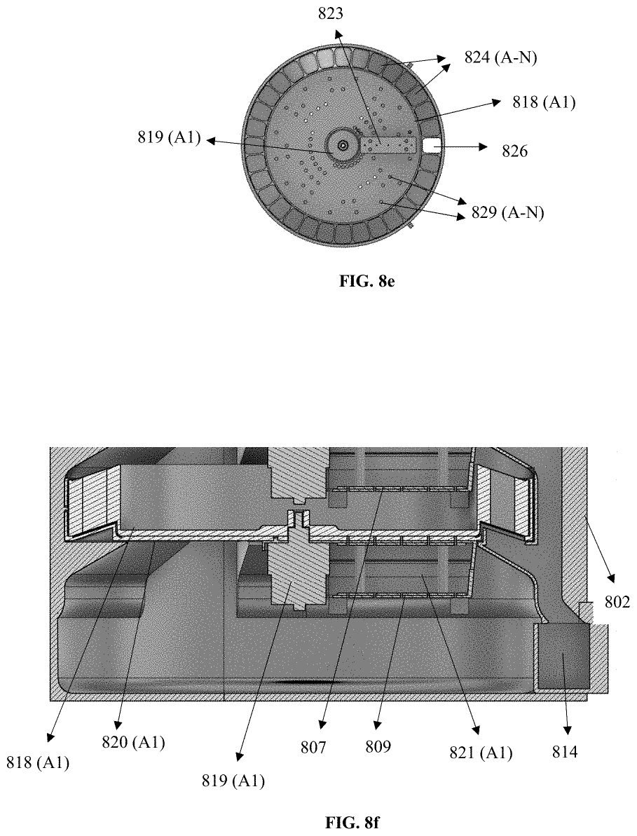

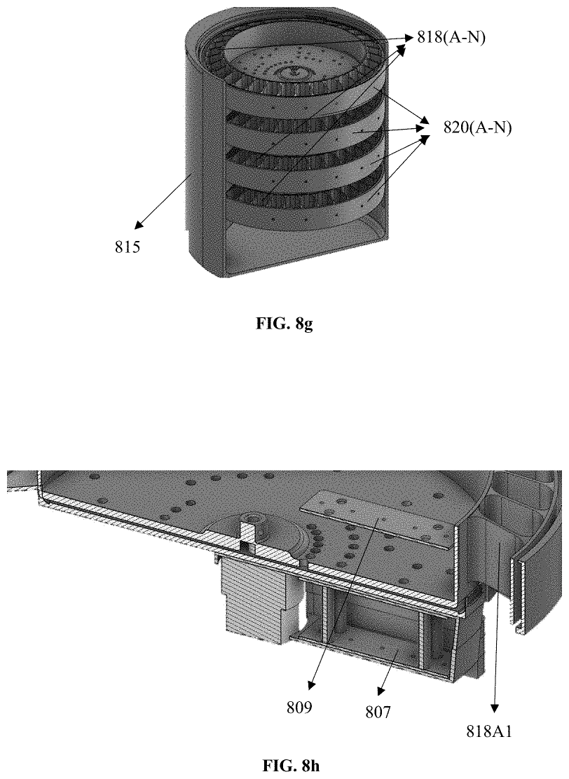

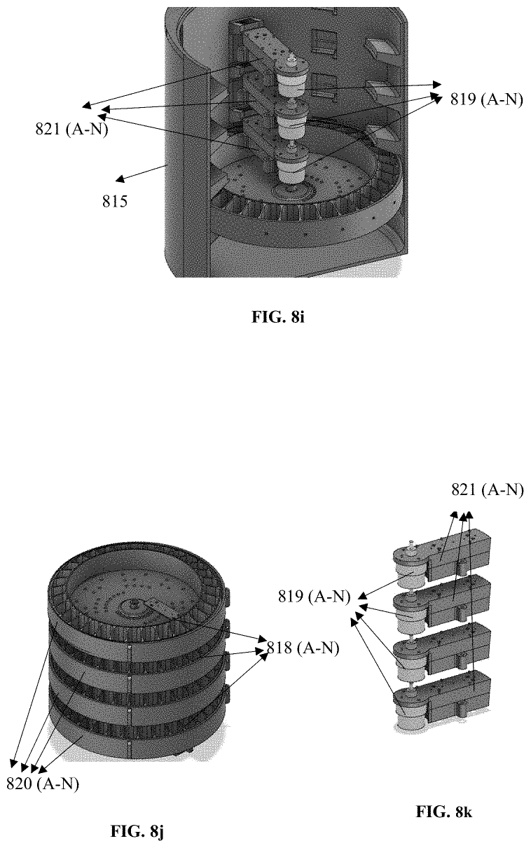

FIG. 8a illustrates a fragmented front view of an outer casing 815 of a handheld device 802, according to one or more embodiments. The outer casing 815 of the handheld device 802 comprises one or more notches 813 A-N and one or more tray holders 811 A-N. A tray holder of the one or more tray holders 811 A-N is adapted to hold a rotatable circular tray. Similarly, a notch of the one or more notches 813A-N is adapted to hold and lock a stationary circular plate in its position.

FIG. 8b illustrates a fragmented cross section of a handheld device 802, according to one or more embodiments. The fragmented cross section shows arrangement of one or more rotatable circular trays 818 (A-N), one or more stationary circular plates 820 (A-N), one or more rotating means 819 (A-N), arrays of identifiers 829 (A-N), a transmitter sensor strip 809, a receiver sensor strip 807, and one or more handles 827 (A-N). In an embodiment, the transmitter sensor strip 809, and the receiver sensor strip 807 are placed interchangeably.

FIG. 8c shows a fragmented cross section of a handheld device 802 with one or more stationary circular plates 820 (A-N) and an outer casing 815, according to one or more embodiments. For an illustrative purpose, FIG. 8c herein shows only the arrangement of the outer casing 815 and the one or more stationary circular plates 820 (A-N). The outer casing 815 comprises one or more notches 813 (A-N). The one or more notches 813 (A-N) are adapted to hold and lock the one or more stationary circular plates 820 (A-N) in position.

FIG. 8d shows a fragmented cross section of a handheld device 802 with one or more stationary circular plates 820 (A-N), one or more rotatable circular trays 818 (A-N), and an outer casing 815, according to one or more embodiments. For an illustrative purpose, FIG. 8d herein shows only the arrangement of the outer casing 815, the one or more stationary circular plates 820 (A-N), and the one or more rotatable circular trays 818 (A-N). The one or more rotatable circular plate 818 (A-N) shows an array of identifiers 829 (A-N) and a plurality of compartments 824 (A-N).

FIG. 8e illustrates a top view of a rotatable circular tray 818 (Al), according to one or more embodiments. The top view depicted herein clearly shows a plurality of compartments 824 (A-N) and an empty compartment out of the plurality of compartments 824 (A-N). The empty compartment is aligned with respect to an aperture 826 of a stationary circular plate once the plurality of compartments 824 (A-N) is filled with medication or loaded in a handheld device. FIG. 8e further depicts a top plate 823 of an arm that couples the rotatable circular tray 818 (Al) with a rotating means 819 (Al) (e.g. PMDC motor, stepper motor, servo motor, etc.). The top view further shows a transmitter sensor strip, affixed on top of the arm, is aligned with respect to the aperture 826, when the empty compartment is aligned with respect to the aperture 826. The top view also depicts an array of identifiers 829 (A-N) on the rotatable circular tray.

FIG. 8f is a fragmented cross section of a bottom most part of a handheld device 802, according to one or more embodiments. FIG. 8f depicts a fragmented view of an arm 821 (Al), a rotating means 819 Al (e.g. a permanent magnet direct current (PMDC) motor), a rotatable circular tray 818 Al, a stationary circular plate 820 Al, a collection tray 814, a transmitter sensor strip 809, and a receiver sensor strip 807.

FIG. 8g depicts a back view of a fragmented cross section of a handheld device 802 illustrating arrangement of one or more rotatable circular trays 818 (A-N), and one or more stationary circular plates 820 (A-N), according to one or more embodiments. The fragmented view in FIG. 8g shows that the one or more rotatable circular trays 818 (A-N) and the one or more stationary circular plates 820 (A-N) are held within an outer casing 815 of the handheld device 802. The outer casing 815 of the handheld device holds the one or more rotatable circular trays 818 (A-N) and the one or more stationary circular plates 820 (A-N) in such a way that the one or more rotatable circular trays 818 (A-N) rotates on top of the one or more stationary circular plates 820 (A-N). The one or more rotatable circular trays 818 (A-N) is released manually or disassembled from the handheld device 802 while refilling the medication. In an embodiment, an outer casing is removed from back side and the medication is filled manually by a pharmacist or a nurse without releasing or disassembling the one or more rotatable circular trays 818 (A-N) from the handheld device 802.

FIG. 8h shows a fragmented cross section of a rotatable circular tray 818 (Al) illustrating arrangement of a transmitter sensor strip 809 and a receiver sensor strip 807, according to one or more embodiments. The fragmented cross section clearly shows that the position of the transmitter sensor strip 809 and the receiver sensor strip 807 with respect to the rotatable circular tray 818 (Al). In an embodiment, the transmitter sensor strip 809, and the receiver sensor strip 807 are arranged interchangeably.

FIG. 8i depicts a fragmented cross section illustrating arrangement of one or more rotating means 819 (A-N) and one or more arms 821 (A-N) inside a handheld device 802, according to one or more embodiments. The fragmented cross section illustrates that a rotatable circular tray 818 (Al) is coupled to the rotating means 819 (Al) through the arm 821 (Al).

FIG. 8j depicts a handheld device 802 without an outer casing, according to one or more embodiments. The handheld device 802 shown herein comprises four set of medication dispenser (i.e. four rotatable circular trays and four stationary circular plates). In an embodiment, the handheld device 802 may comprise any number of rotatable circular trays and stationary circular plates as per the medication period and medication timings.

FIG. 8k illustrates arrangement of one or more rotating means 819 (A-N), and one or more arms 821 (A-N), according to one or more embodiments. The one or more rotating means 819 (A-N) are aligned linearly. In an embodiment, the one or more rotating means 819 (A-N) are coupled via a common shaft. The one or more rotating means 819 (A-N) are mechanically coupled with the one or more arms 821 (A-N) such that the when the one or more rotating means 819 (A-N) rotates, the one or more arms 821 (A-N) also moves simultaneously. The one or more rotating means 819 are capable of rotating individually.

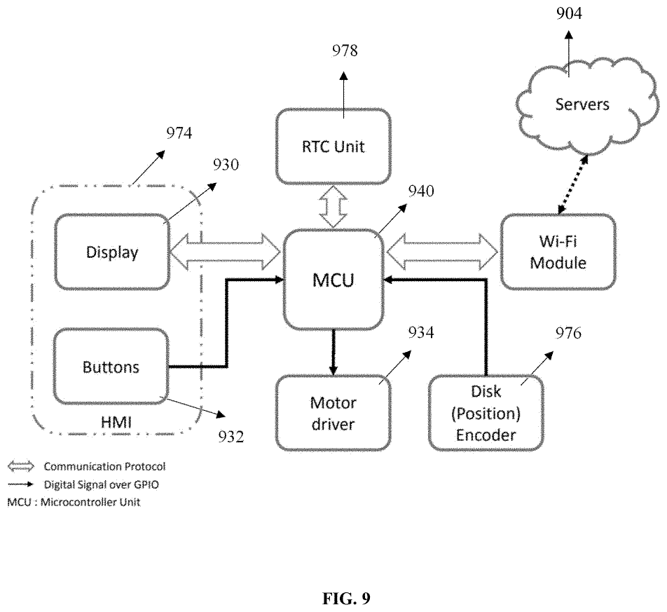

FIG. 9 illustrates a block diagram of a handheld device 902, according to one or more embodiments. The handheld device comprises a microcontroller unit (MCU) 940, a human machine interface (HMI) 974, a motor driver unit 934, a disk position encoder 976, a real-time clock (RTC) unit 978, and a wireless connectivity module 980. The microcontroller unit (MCU) 940 provides instruction to units (e.g. components) associated within and outside the handheld device 902. The microcontroller unit (MCU) 940 may receive and provide information and signal through the human machine interface (HMI) 974. The human machine interface (HMI) 974 may comprise a display 930 and an input unit 932 (e.g. keyboard, buttons).

The input unit is adapted to provide input to the handheld device 902. The motor driver unit 934 is configured to control and provide excitation to one or more rotating means. The one or more rotating means comprise one of (a) a permanent magnet direct current (PMDC) motor, (b) a stepper motor, and (c) a servo motor to actuate one or more rotatable circular trays to align a plurality of compartments of a rotatable circular tray with respect to an aperture in a stationary circular plate at a scheduled time. In an embodiment, the motor driver unit 934 comprises one or more Semiconductor switches (Integrated Circuits-ICs) to control at least one rotating means (e.g. PMDC motor, stepper motor, servo motor)

The disk position encoder 976 is configured to determine position or alignment of the rotatable circular tray with respect to the aperture of the stationary circular plate. The disk position encoder comprises a transmitter sensor strip and the receiver sensor strip. In an embodiment, the rotatable circular tray is positioned between the transmitter sensor strip and a receiver sensor strip. The transmitter sensor strip and the receiver sensor strip may comprise equal number of sensors. An array of identifiers is arranged in one or more patterns on the rotatable circular tray based on count of one or more transmitter sensors in the transmitter sensor strip and one or more receiver sensors in the receiver sensor strip. The transmitter sensor strip may transmit at least one signal to the receiver sensor strip. As the rotatable circular tray comprising the array of identifiers is placed between the transmitter sensor strip and the receiver sensor strip, the signal communicated from the transmitter sensor strip to the receiver sensor strip is interrupted. For an instance, the transmitter sensor strip comprises five transmitter sensors and the receiver sensor strip comprises five receiver sensors. In this case, the array of identifiers may be arranged in 2\hat{ }5=32 different patterns to identify 32 different alignments or position of the rotatable circular tray with respect to an aperture. The one or more patterns may comprise at least one identifier of the array of identifiers arranged in one or more positions and in one or more counts with respect to the one or more transmitter sensors and the one or more receiver sensors. The signal communicated from the transmitter sensor strip to the receiver sensor strip flows only through the array of identifiers that are aligned with respect to the one or more transmitters sensors and the one or more receive sensors. The rest of the identifiers interrupts the signal. Therefore, based on the position and the count of the identifiers arranged in that pattern, the disk position encoder 976 determines the alignment and position of the rotatable circular tray with respect to the aperture. The alignment or position of the rotatable circular tray with respect to at least one pattern is adapted to estimate a medication day (e.g. first day, second day, etc.) of a medication period.

The real-time clock (RTC) unit 978 is configured to keep track of current timing and the scheduled time. The real-time clock (RTC) unit 978 is further configured to communicate a signal to the microcontroller unit (MCU) 940 to trigger signal and communicate alerts, signals, data and notifications at respective timings. The real-time clock (RTC) unit 978 keeps track the of current timing and the scheduled time both when the handheld device 902 is ON and when the handheld device 902 is OFF. The wireless connectivity module 980 is configured to communicate with at least one of a computing unit and a server. The handheld device 902 may further comprise buzzers to alert, notify, and remind at the scheduled time.

The handheld device 902 further comprises a power management module, a camera system, a sensors module, a memory module, and a communication module. The power management module is configured to supply excitation to electronic components associated with the handheld device 902. In an embodiment, the power management module is rechargeable and battery powered. In another embodiment, the power management module is directly connected to an electric supply (i.e. like a plug-in device).

The camera system is configured to operate for a third predefined period of time during when the handheld device 902 dispenses the medication into a collection tray. The third predefined period of time may range from twenty to thirty minutes from the scheduled time. In an embodiment, the third predefined period of time is customizable via a user interface. The camera system captures at least one of an image, and a video around a scene of the handheld device 902, around the collection tray, to ensure whether the patient (i.e. intended user) is collecting and consuming the medication. In another embodiment, the camera system is communicatively coupled with an artificial intelligence-based image processing unit to analyze at least one face captured in the image and video, and determine whether the at least one face belongs to the patient to provide accessibility and ensure medication compliance. In yet another, the camera system is communicatively coupled with an artificial intelligence-based voice processing unit to analyze at least one voice input received from the patient and determine whether the at least one voice input belongs to the patient to provide accessibility and ensure medication compliance.

The sensors module comprises a physiological sensor, an identification sensor, etc. at one or more places in the handheld device 902. In an embodiment, the sensors module comprises the physiological sensor, and the identification sensor that are placed integrated. The sensors module acquires data (e.g. physiological signal, identification data, etc.) and communicates the data to the server. The memory module stores voice inputs, medication name, medication dosage, medication timings, etc. locally. The memory module stores one or more instructions that are to be executed by the handheld device 902. The communication module communicates data (e.g. the physiological signal, the identification data, etc.) and signal to the server and the computing unit. The communication module is further configured to receive a dispensing trigger signal and data from the server. The dispensing trigger signal is adapted to start dispense the medication from the plurality of compartments at the scheduled time. In an embodiment, the micro controller, upon receiving the dispensing trigger signal, instructs the one or more rotating means to actuate and dispense the medication.

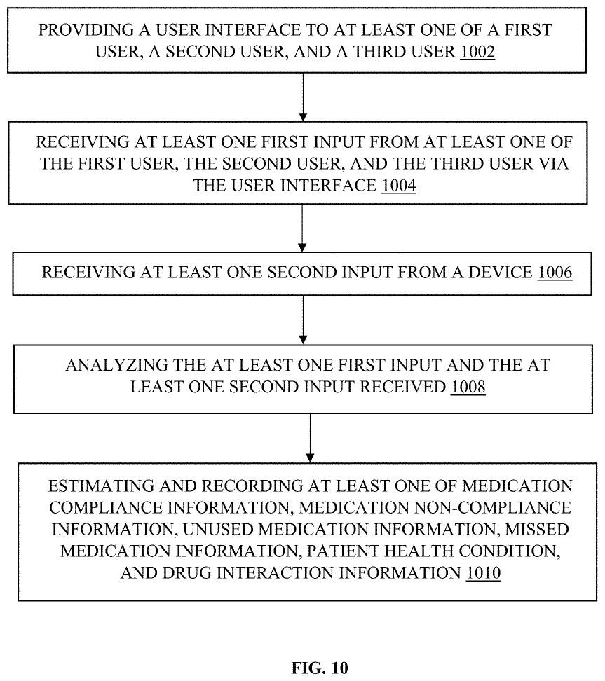

FIG. 10 illustrates a method of tracking and ensuring medication compliance, according to one or more embodiments. At step 1002, a user interface is provided, by a server, to at least one of a first user, a second user, and a third user. At step 1004, at least one first input is received from at least one of the first user, the second user, and the third user via the user interface. The at least one first input may comprise medication information, medication timing information, patient information, a prescription, and a voice alert input. At step 1006, at least one second input is received from the handheld device. The at least one second input comprises collection tray operating information, collection tray timing information, disposal tray operating information, disposal tray timing information, and physiological signal. At step 1008, the at least one first input and the at least one second input is analyzed by the server. At step 1010, at least one of medication compliance information, medication non-compliance information, unused medication information, missed medication information, patient health condition, and drug interaction information is estimated and recorded.

The foregoing disclosure provides illustration and description, but is not intended to be exhaustive or to limit the implementations to the precise form disclosed. Modifications and variations are possible in light of the above disclosure or may be acquired from practice of the implementations.

Those skilled in the art will appreciate that the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, etc. The invention may also be practiced in distributed system environments where local and remote computer systems, which are linked (either by hardwired data links, wireless data links, or by a combination of hardwired and wireless data links) through a network, both perform tasks. In a distributed system environment, program modules, units may be located in both local and remote memory storage devices.

The present invention may be embodied in other specific forms without departing from its spirit or characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

Although the present embodiments have been described with reference to specific example embodiments, it will be evident that various modifications and changes may be made to these embodiments without departing from the broader spirit and scope of the various embodiments. For example, the various devices, units and modules described herein may be enabled and operated using hardware circuitry (e.g., CMOS based logic circuitry), firmware, software or any combination of hardware, firmware, and software (e.g., embodied in a non-transitory machine-readable medium). For example, the various electrical structures and methods may be embodied using transistors, logic gates, and electrical circuits (e.g., application specific integrated (ASIC) circuitry and/or Digital Signal Processor (DSP) circuitry).

In addition, it will be appreciated that the various operations, processes, and methods disclosed herein may be embodied in a non-transitory machine-readable medium and/or a system. Accordingly, the specification and drawings are to be regarded in an illustrative rather than a restrictive sense.

### INCORPORATION BY REFERENCE

All patents, patent application publications, and non-patent literature mentioned in the application are incorporated by reference in their entirety.

U.S. Pat. No. 6,439,422 entitled “Automated portable medication radial dispensing apparatus and method”;

U.S. Pat. No. 7,302,311 entitled “Cartridge for dispensing pill- or capsule-form medications in desired doses”;

US20160132660 entitled “Electronic medication adherence, identification, and dispensation”;

U.S. Pat. No. 6,604,019 entitled “Automated pharmaceutical management and dispensing system”;

U.S. Pat. No. 9,717,654 entitled “Electronic pill box prefill system including a blister pack with a capacitive sensor”;

U.S. Pat. No. 8,161,968 entitled “Medicament dispenser”;

U.S. Pat. No. 10,791,881 entitled “Low cost radio frequency identification (RFID) dispensing systems”;

US20200253828 entitled “Automated and secure methods for dispensing medication”;

US20160081883 entitled “Medication cassette and dispensing tracking apparatus”.

What is claimed is:

1. A system comprising:
a server comprising a memory, and a processor communicatively coupled to the memory, the processor operable to
provide a user interface to at least one of a first user, a second user, and a third user;
receive at least one first input from at least one of the first user, the second user, and the third user via the user interface, wherein the at least one first input comprises medication information, medication timing information, patient information, a prescription, a regimen, and a voice alert input;
receive at least one second input from a device, wherein the at least one second input comprises collection tray operating information, collection tray timing information, disposal tray operating information, disposal tray timing information, and physiological signal;
analyze the at least one first input and the at least one second input received; and
estimate and record at least one of medication compliance information, medication non-compliance information, unused medication information, missed medication information, patient health condition, and drug interaction information.

2. The system of claim 1, wherein the server communicates with a drug database to extract at least one of the drug interaction information, side effects information associated with at least one medication, and composition information of the at least one medication.

3. The system of claim 1, wherein the user interface enables interaction between the first user, the second user, and the third user.

4. The system of claim 1, wherein the server is operable to customize the voice alert input based on the at least one first input.

5. The system of claim 2, wherein the server is operable to communicate with the drug database and extract a medication procedure based on the patient health condition.

6. The system of claim 1, wherein the server is operable to notify the medication compliance information, the medication non-compliance information, the unused medication information, and the missed medication information to at least one of the first user, the second user, and the third user.

7. A non-transitory computer storage medium storing a sequence of instructions, which when executed by a processor, causes:
provide a user interface to at least one of a first user, a second user, and a third user;
receive at least one first input from at least one of the first user, the second user, and the third user via the user interface, wherein the at least one first input comprises medication information, medication timing information, patient information, a prescription, a regimen, and a voice alert input;
receive at least one second input from a device, wherein the at least one second input comprises collection tray operating information, collection tray timing information, disposal tray operating information, disposal tray timing information, and physiological signal;
analyze the at least one first input and the at least one second input received; and
estimate and record at least one of medication compliance information, medication non-compliance information, unused medication information, missed medication information, patient health condition, and drug interaction information.

8. The system of claim 1, wherein the server is operable to create a trigger signal and communicate the trigger signal to the device.

9. The system of claim 1, wherein the server is operable to create at least one of graphs and charts of at least one medication of at least one patient upon receiving the at least one first input.

10. The system of claim 1, wherein the server is operable to realize present time and keep track of a scheduled time.

11. The system of claim 1, wherein the server is operable to enable the first user to request an access code for a secondary medication container from the third user.

12. The system of claim 11, wherein the server is operable to disassemble the secondary medication container from the device upon inputting the access code.

13. The system of claim 12, wherein the server tracks and records the access code.

14. The system of claim 13, wherein the server tracks and records as-needed medication information, dosage information of the as-needed medication, the unused medication information, the missed medication information, the medication compliance information, and the medication non-compliance information.

15. The system of claim 1, wherein the server is further operable to:
store the medication information, the patient information, side-effects of at least one medication, medication dosage in a database.

16. The system of claim 1, wherein the server is further operable to:
enable the second user to remotely operate at least one sensor in the device; and
acquire at least one physiological signal from the first user.

17. The system of claim 8, wherein the trigger signal is adapted to start dispense at least one medication from a plurality of compartments of the device at a scheduled time.

18. The system of claim 11, wherein the server is operable to operate and dispense at least one medication from the secondary medication container upon inputting the access code.

19. The system of claim 1, wherein the server is operable to alert and remind at least one of the first user, the second user and the third user at a predefined time from a scheduled time.

20. The system of claim 1, wherein the device comprises (a) a display that depicts at least one of the medication information, the medication timing information, the patient information, a medication procedure, and (b) a speaker that alerts and guides the first user at a scheduled time.
